(19) United States
(12) Patent Application Publication
(10) Pub. No.: US 2016/0067082 A1
(43) Pub. Date: Mar. 10, 2016

(54) MICRO-BALLOON VITREOUS SEPARATOR

(71) Applicant: KHALED KHALEEL SABTI, SAFAT (KW)

(72) Inventor: KHALED KHALEEL SABTI, SAFAT (KW)

(21) Appl. No.: 14/481,887

(22) Filed: Sep. 9, 2014

(51) Int. Cl. A61F 9/007; A61M 29/02

(52) U.S. Cl. CPC A61F 9/007; A61M 29/02; A61F 2250/0007

(57) ABSTRACT

The micro-balloon vitreous separator generally includes a handle, an elongated shaft coupled to an end of the handle, a tube, and a control device. The tube has an inflatable member and a tip at a distal portion of the tube. The proximal portion of the tube is configured for attachment to an inflation medium. The control device includes a gripping member that is disposed within the handle and engages at least a portion of the tube, and a toggle member that extends through a slot in the handle. The toggle member of the control device may be manipulated by a user to extend and retract the distal portion of the tube. The tip of the tube can be positioned between the vitreous humor and the retina and the inflatable member can be inflated to separate the vitreous humor from the retina.

### BACKGROUND OF THE INVENTION

1. Field of the Invention

The present invention relates to surgical devices, and, more specifically to a micro-balloon vitreous separator for separating the vitreous humor from the retina of an eye.

2. Description of the Related Art

One of the factors that play an important role in a successful retinal surgery is the relationship between the retina and the vitreous humor. The vitreous humor is the transparent, gel like substance that fills the space between the lens and the retina of an eyeball. As such, it is important to separate the vitreous humor from the retina before a surgeon can operate on the retina. However, strong adhesions between the retina and the vitreous humor make the separation of the vitreous humor and the retina difficult.

Typically, the vitreous humor and the retina can be separated mechanically. One manner in which the vitreous humor and the retina are separated is through the use of high suction and a vitrectomy cutter. Another way is through the use of micro-scissors, a mirco-spatula, and a micro-diamond knife. A bimanual surgical technique using these instruments has resulted in higher success rates of retinal re-attachments through complete separation of the vitreous humor from the retina. Bimanual retinal surgery is frequently used in difficult cases typically because advanced light sources allow the use of light through a fixed light system to enable a surgeon to use both hands freely. The surgeon would typically hold the micro-forceps in one hand and another instrument, such as micro-scissors, in the other hand. The micro-forceps are used to pull the vitreous humor away from the retina. Pulling away of the vitreous humor in this manner is possible when the retina is attached, i.e., fixed in position, as counterforces hold the retina in place while the vitreous humor is pulled away and separated from the retina.

However, in certain situations, such as rhegmatogenous retinal detachments and tractional retinal detachments, where the retina is detached and freely mobile, counterforces do not exist to aid in separation of the vitreous humor from the retina, thereby making mechanical separation using micro-forceps extremely difficult, if not impossible.

Thus, a micro-balloon vitreous separator addressing the aforementioned problems is desired.

### SUMMARY OF THE INVENTION

The micro-balloon vitreous separator generally includes a handle, an elongated shaft coupled to an end of the handle, a tube, and a control device. The handle has a diameter greater than the diameter of the elongated shaft. The tube has a proximal portion, a distal portion, a lumen, an inflatable member, and a tip at the distal portion of the tube. The proximal portion of the tube is configured for attachment to an inflation medium. The control device includes a gripping member that is disposed within the handle and a toggle member that extends through a slot in the handle. The tube extends through the micro-balloon vitreous separator with at least a portion thereof extending through the gripping member.

The toggle member of the control device may be manipulated by a user to extend and retract the distal portion of the tube. The tip of the tube can be positioned between the vitreous humor and the retina. The inflatable member of the tube can be inflated to separate the vitreous humor from the retina. After separating the vitreous humor from the retina, and deflating the inflatable member, the control device can be used to retract the distal end of the tube so as to remove the micro-balloon vitreous separator from the eye.

These and other features of the present invention will become readily apparent upon further review of the following specification and drawings.

### BRIEF DESCRIPTION OF THE DRAWINGS

Fig, 1 is an environmental, perspective view of a micro-balloon vitreous separator according to the present invention.

FIG. 2 is a top perspective view of a handle associated with a control device according to the present invention.

FIG. 3 is a semi-exploded view of the handle shown in FIG. 2.

FIG. 4 is a sectional side view of an eye.

FIG. 5 is a sectional side view of the micro-balloon vitreous separator having an inflated inflatable member separating the vitreous humor from the retina of the eye according to the present invention.

FIG. 6 is an alternative sectional side view of the micro-balloon vitreous separator having an inflated inflatable member separating the vitreous humor from the retina of the eye according to the present invention.

FIG. 7 is an alternative sectional side view of the micro-balloon vitreous separator having an inflated inflatable member separating the vitreous humor from the retina of the eye according to the present invention,

Similar reference characters denote corresponding features consistently throughout the attached drawings.

### DETAILED DESCRIPTION OF THE PREFERRED EMBODIMENTS

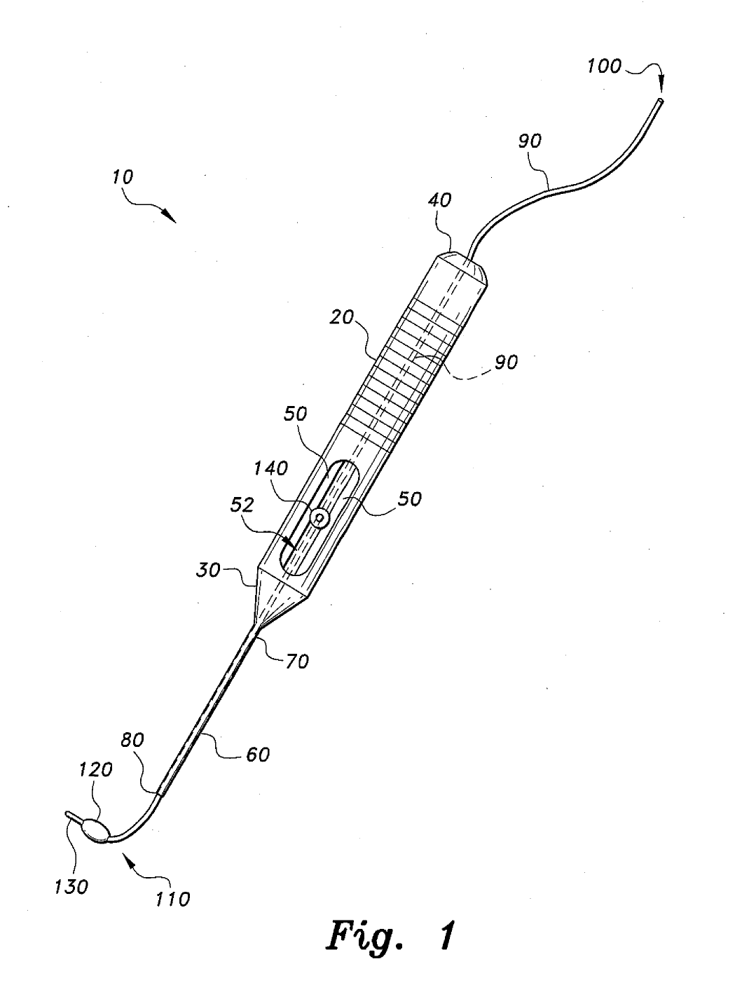

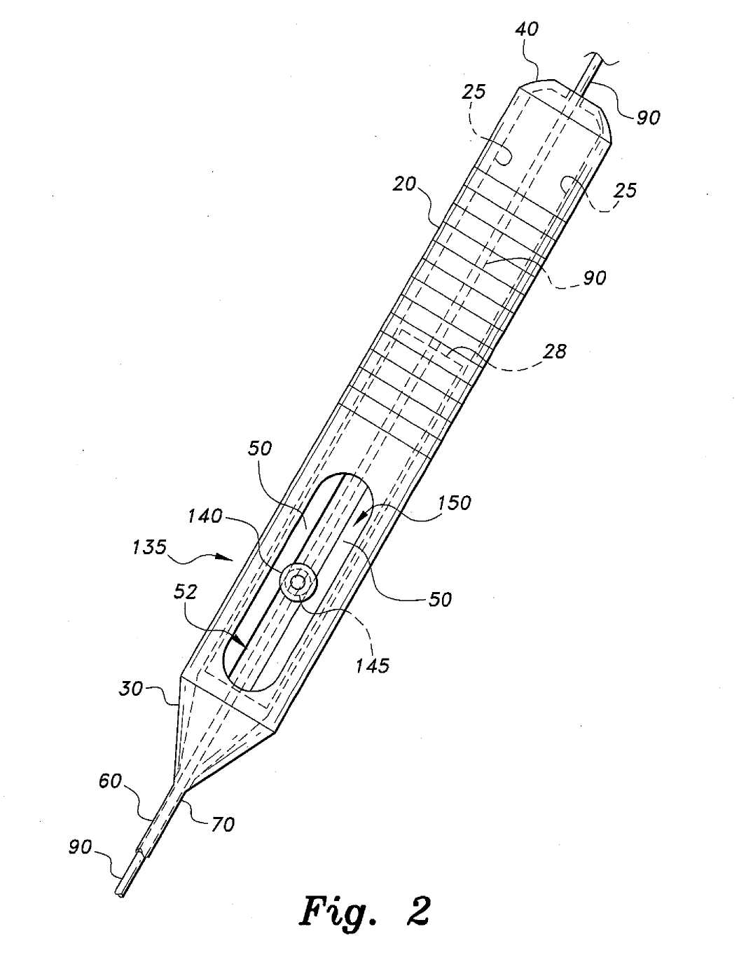

Referring to FIGS. 1 and 2, an embodiment of a micro-balloon vitreous separator, generally identified as 10, is illustrated. The micro-balloon vitreous separator 10 generally includes a handle 20 having an anterior end 30, a posterior end 40, and a cylindrical wall 25 extending from the anterior end 30 to the posterior end 40. The handle 20, having a given diameter, is coupled to an elongated shaft 60 having a diameter smaller than the diameter of the handle 20. The elongated shaft 60 includes a proximal end 70, a distal end 80, and a lumen extending from the proximal end 70 to the distal end 80. The micro-balloon vitreous separator 10 further includes a tube 90 and a control device 135 in communication with the tube 90. The tube 90 has a proximal portion 100, a distal portion 110, a lumen, an inflatable member 120, and a tip 130. The control device 135 is accessible to a user through an elongated slot 150 in the handle 20. The control device 135 has a toggle member 140 extending through the slot 150. The control device 135 may be manipulated by a user to extend and retract the distal portion 110 of the tube 90.

The handle 20 can include two protrusions or guides 50 extending from opposing positions of the cylindrical wall 25. The guides 50 can be configured to extend along the length of the slot 150, as shown, or along the length of the handle. A channel 52 can be formed by a space or opening between the two guides 50. The anterior end 30 of the handle 20 can be adapted to receive the proximal end 70 of the elongated shaft 60. The posterior end 40 of the handle 20 can have an opening (not shown) through which the tube 90 can be inserted into the handle 20.

The handle 20 can be formed from a suitable medical grade material, such as medical grade plastic, for example. While the shape of the handle 20 can vary, it is desirable that the handle 20 have an ergonomic shape, for example. It is to be noted that the length and the diameter of the handle 20 can also vary. It is desirable, however, that the handle 20 have a length of at least 80 mm and a diameter of at least 15 mm.

The elongated shaft 60 can be formed of any suitable medical grade material and can vary in length, as well as in diameter. The elongated shaft 60 can include a wall of substantially uniform thickness having an outer surface and an inner surface. The lumen within the elongated shaft 60 is adapted to receive a medical implement, such as the tube 90, therein. It is desirable that the elongated shaft 60 include a hollow, metallic shaft having a length of at least 30 mm and a 20 gauge diameter. The proximal end 70 of the elongated shaft 60 is in communication with the anterior end 30 of the handle 20.

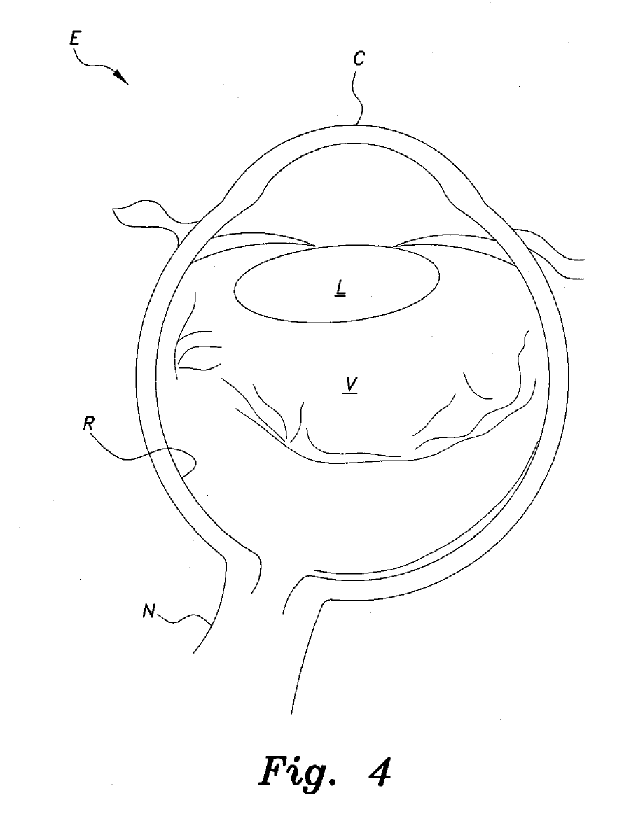

The tube 90 can be formed from any suitable, medical grade material, such as silicon, for example, and is preferably a flexible tubular member formed from polymethyl methacrylate. The tube 90 can include a wall of substantially uniform thickness having an outer surface and an inner surface. It is to be noted that while the tube 90 can vary in length and width, it is desirable that the tube 90 have a length and a width suitable to extend through the handle 20 and the lumen of the elongated shaft 60 and reach the retina R of an eye E (FIGS. 4-7).

The proximal portion 100 of the tube 90 is adapted to receive an inflation medium, such as air, from an inflation source, such as an air pressure source, to facilitate inflation of the inflation member 120. The distal portion 110 of the tube 90 can include the inflatable member 120 and the tip 130. The tip 130 can have a blunt edge, e.g., spatula-shaped, to facilitate separation of the vitreous humor V from the retina R of the eye E (FIGS. 4-7).

The inflatable member 120 can be a micro balloon, for example. The inflatable member, 120 can be formed from any suitable expandable material that is strong and puncture-resistant, e.g., polyethylene terephthalate (PET), nylon, polyurethane, and/or other elastomers. The inflatable member 120 can also be adapted to expand to a specific size. The inflatable member 120 can be produced in a wide range of diameters, lengths, and shapes, such as conical, spherical, and tapered, for example. It is desirable, however, that the inflatable member 120 have a length of about 4 mm to 12 mm and a spherical shape adapted to separate the vitreous humor V from the retina R of the eye E (FIGS. 4-7). The inflatable member 120 can also be coated for lubrication or for abrasion resistance.

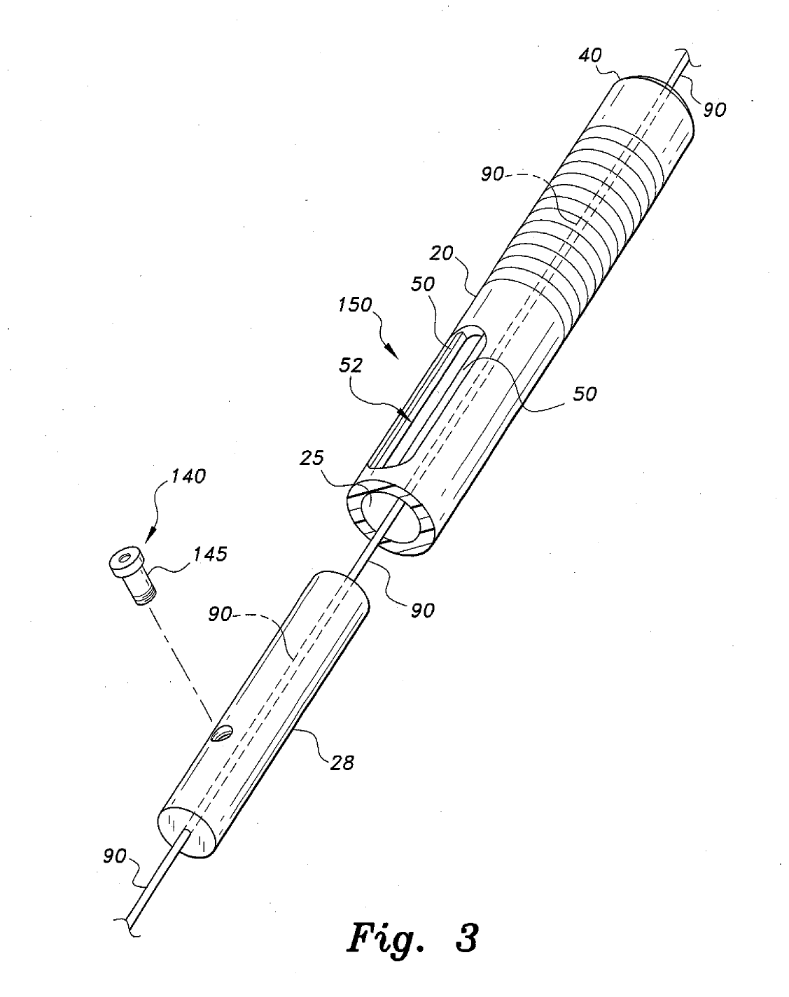

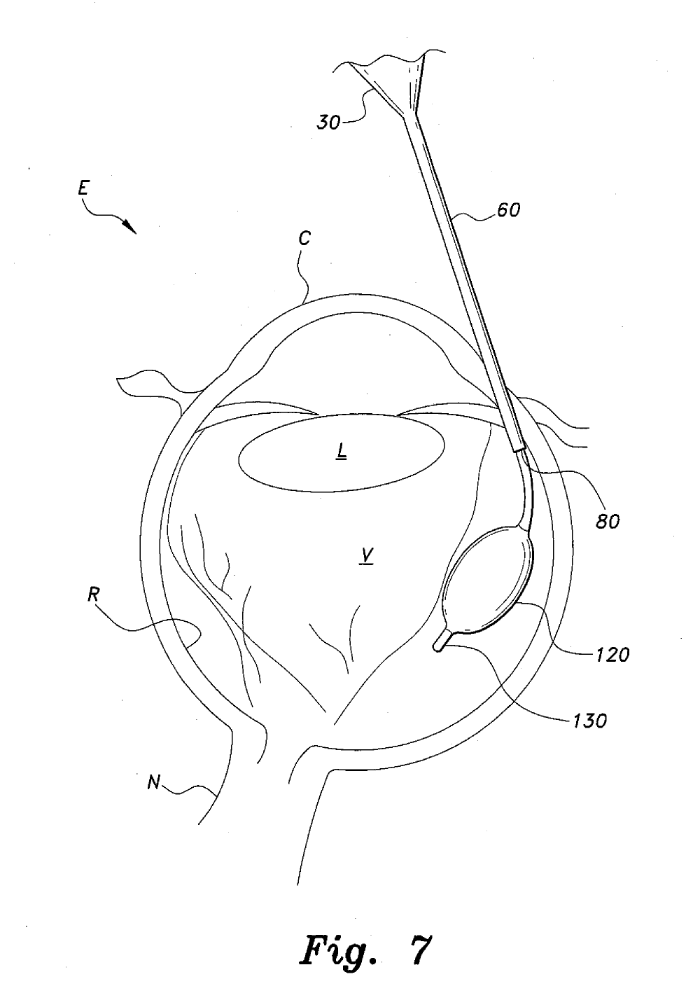

As shown more clearly in FIG. 3, the toggle member 140 is connected to gripping member 28. The gripping member 28 has an opening through which at least a portion of the tube 90 may extend. The diameter of the opening of the gripping member 28 is configured to be only slightly larger than the diameter of the tube 90, such that the gripping member 28 may “grip” or engage at least a portion of the tube 90. The toggle member 140 may include a button or other suitable structure which may easily be moved within the slot 150. A base portion 145 of the toggle member 140 may abut a surface of the guides 50, thereby providing sufficient friction or a friction locking mechanism (discussed below) to avoid unintended sliding of the toggle member 140 within the channel 52. The base portion 145 may be threaded. The toggle member 140 may be moved by a user to simultaneously move the gripping member 28 and the tube 90 positioned within the gripping member 28. The tube 90 may, thereby, move in unison with the gripping member 28 when the toggle member 140 is manipulated by a user to extend and retract the distal portion 110 of the tube 90. As such, movement of the gripping member 28 towards the distal portion 110 of the tube 90 can move the inflatable member 120 towards the retina R of the eye E (FIGS. 4-7), while movement of the gripping member 28 towards the proximal portion 100 of the tube can retract the tube 90, e.g., when the vitreous humor V (FIGS. 4-7) and the retina R (FIGS. 4-7) have been separated, as illustrated in FIG. 7.

As mentioned above, the gripping member 28 can travel in a forward direction and in a backward direction along the channel 52. As the toggle member 140, in communication with the gripping member 28, is moved in a forward direction along the channel 52, the proximal portion 110 of the tube 90 having the inflatable member 120 and the tip 130 is extended beyond the proximal end 80 of the elongated shaft 60. As the toggle member 140, in communication with the gripping member 28, is moved in a backward direction along the channel 52, the proximal portion 110 of the tube 90 is retracted back into the elongated shaft 60. The control device 135 can be adapted to retract about 30 mm of the tube 90. It is to be noted that the control device 135 can also be adapted to include a locking mechanism, such as a friction locking mechanism, for example. The interior portion of the guides 50 can contact the base 145 of the toggle member 140, which can form a friction locking mechanism to prevent the gripping member 28 and the tube 90 from moving freely in a forward or backward direction.

Referring to FIGS. 1-7, in operation, the distal end 110 of the tube 90 having the inflatable member 120 and the tip 130 can be inserted through the opening of the gripping member 28. The gripping member 28 may then be inserted through the opening (not shown) of the handle, located at the posterior end 40 of the handle 20, The gripping member 28 may slidably engage an interior surface of the cylindrical wall 25 and direct the distal end 130 of the tube 90 through the lumen of the elongated shaft 60. Once the gripping member 28 is positioned in the handle 20, the toggle member can be screwed into a corresponding aperture in the gripping member 28.

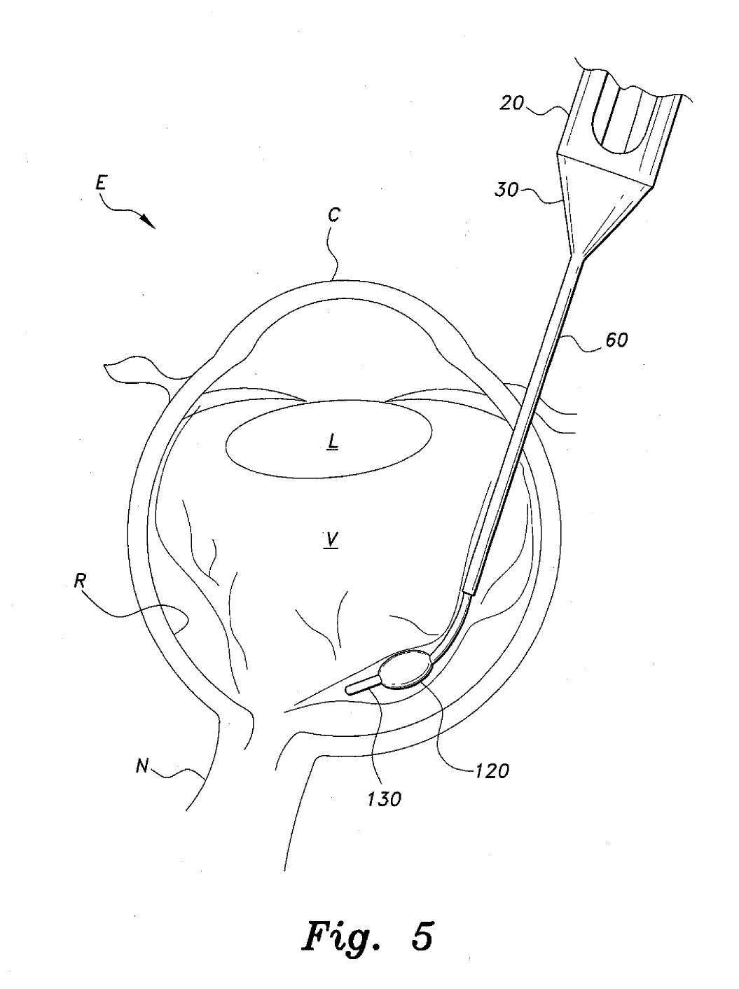

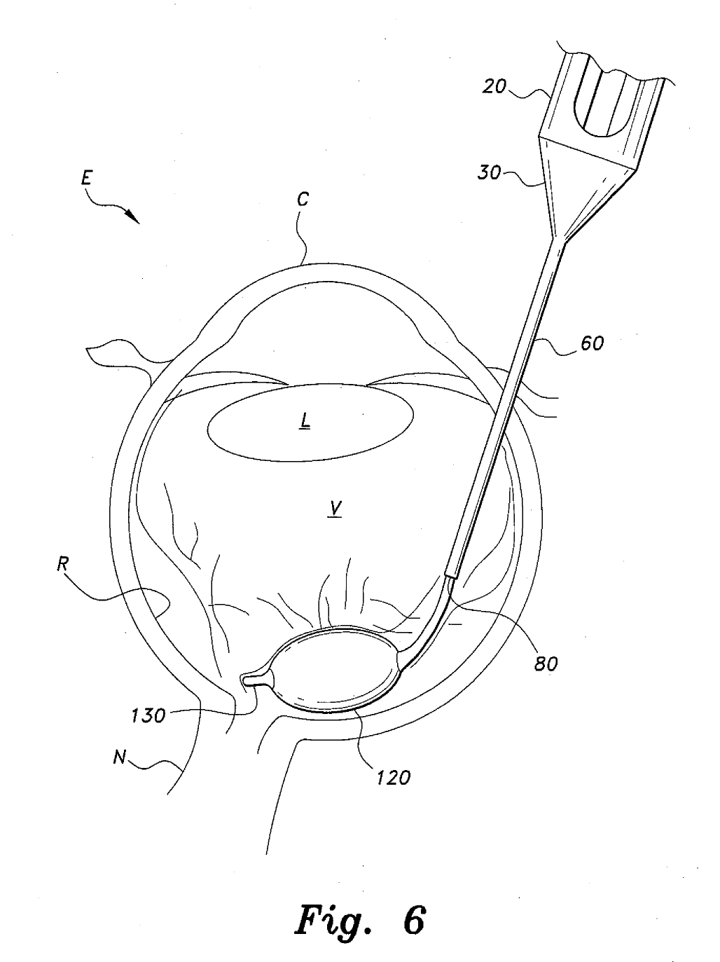

The micro-balloon vitreous separator 10 can be used to separate the vitreous humor V from a detached retina with little or no trauma. Once the tube 90 is properly positioned within the micro-balloon vitreous separator 10, the surgeon can introduce the tip 130 of the tube 90 to an appropriate space between the vitreous humor V and the retina R to create a space therebetween, as illustrated in FIG. 5. The inflatable member 120 can be inflated gently to detach the vitreous humor V from the retina R. As the inflatable member 120 expands uniformly in all directions to pull the vitreous humor V away, counter forces on the retina are not needed. Referring to FIGS. 6-7, by inflating the inflatable member 120, the micro-balloon vitreous separator 10 can be progressively advanced to non-separated areas of the vitreous humor V and the retina R until the appropriate amount of retina R has been exposed. The inflatable member 120 can be inflated and deflated several times until the two layers are completely separated. Once the appropriate amount of retina R has been exposed, the inflatable member 120 is deflated and the toggle member 140 of the control device 135 (FIG. 2) can be used to retract the tube 90 into the elongated shaft 60. After the tube 90 has been retracted into the elongated shaft 60, the micro-balloon vitreous separator 10 can be removed from the eye.

It is to be understood that the present invention is not limited to the embodiments described above, but encompasses any and all embodiments within the scope of the following claims.

1-8. (canceled)

9. A micro-balloon vitreous separator, consisting of:
an elongated handle consisting of an anterior end, a posterior end, a cylindrical wall extending from the anterior end to the posterior end, and a continuous longitudinal slot formed through the cylindrical wall, the slot defining opposed ends and including a pair of guides, each of the guides protruding from and located below a peripheral surface of the cylindrical wall along the length of the slot and terminating at opposed ends;
an elongated shaft having a proximal end, a distal end, and a lumen extending through the elongated shaft, the elongated shaft coupled to the anterior end of the handle and having a diameter less than a diameter of the handle;
a tube having a proximal portion, a distal portion including an inflatable member and a tip, and a lumen extending through the tube, wherein the tip defines a blunt, spatula-shaped edge for separating the tissues of the retina and the vitreous humor of an eye;
a control device having a gripping member and a toggle member, the toggle member being moved by a user to extend or retract the distal portion of the tube, the gripping member disposed solely in the handle and engaging at least a portion of the tube, and the toggle member connected to the gripping member, the toggle member consisting of a base portion extending through the slot of the handle, the base portion being sized and configured to engage the guides thereby forming a friction lock therewith.

10-16. (canceled)
